(19) United States
(12) Patent Application Publication
(10) Pub. No.: US 2024/0173360 A1
(43) Pub. Date: May 30, 2024

(54) COMPOSITIONS AND METHODS FOR TREATING EPILEPSY AND RELATED DISORDERS

(71) Applicant: Finch Therapeutics Holdings LLC, Boston, MA (US)

(72) Inventor: Thomas Julius Borody, Five Dock (AU)

(73) Assignee: Finch Therapeutics Holdings LLC, Boston, MA (US)

(21) Appl. No.: 18/425,385

(22) Filed: Jan. 29, 2024

Related U.S. Application Data
(63) Continuation of application No. 17/280,708, filed on Mar. 26, 2021, now Pat. No. 11,911,419.
(60) Provisional application No. 62/737,477, filed on Sep. 27, 2018.

(51) Int. Cl. A61K 35/741; A61K 9/00; A61K 9/48; A61K 31/4164; A61K 35/24; A61K 38/14; A61P 25/08

(52) U.S. Cl. CPC A61K 35/741; A61K 9/0053; A61K 9/48; A61K 31/4164; A61K 35/24; A61K 38/14; A61P 25/08

(57) ABSTRACT

The present disclosure is in the field of pharmaceutical compositions suitable for the treatment of diseases in mammals. The disclosure provides novel compositions comprising non-pathogenic fecal microbes for treating epilepsy, epileptic seizures and related diseases. The disclosure also provides methods for treating a subject with the compositions disclosed herein.

### CROSS-REFERENCE TO RELATED APPLICATIONS

This application is a continuation of U.S. patent application Ser. No. 17/280,708, filed Mar. 26, 2021, which is a U.S. National Stage of International Application No. PCT/US2019/053400, filed Sep. 27, 2019, which claims the benefit of U.S. Provisional Application No. 62/737,477, filed Sep. 27, 2018, all of which are incorporated by reference herein in their entireties.

### FIELD

The present disclosure relates to pharmaceutical compositions and methods suitable for treating epilepsy.

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and results in increased susceptibility to pathogens, altered metabolic profiles, or induction of proinflammatory signals that can lead to local or systemic inflammation or autoimmunity. Additionally, such a disrupted microbiota may be infected by incoming pathogen or pathogens, which can cause pain, diarrhea, gas, and constipation among other symptoms. Hence, the intestinal microbiota plays a significant role in the pathogenesis of many disorders such as pathogenic infections of the gut.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal or nasogastric tube of human microbiota either in the form of homogenised stool, or cultured stool components such as Clostridia, to implant in the colon and thereby displace or eradicate pathogenic bacteria, e.g., C. difficile.

Without being bound to any theory, epilepsy is considered by some as a central nervous system disorder in which nerve cell activity in the brain is disturbed, causing seizures. During a seizure, a person experiences abnormal behavior, symptoms, and sensations. A person may also lose consciousness during and after a seizure. Epilepsy may occur as a result of a genetic disorder or an acquired brain injury, including trauma, brain tumor, central nervous system infection, or stroke. People are diagnosed with epilepsy when they have experienced two or more seizures. Seizures are classified into two groups, generalized and focal seizures. A generalized seizure affects both sides of the brain and can be characterized as absence, tonic, atonic, clonic, myoclonic, or tonic-clonic seizures. The absence seizure, also known as a petit mal seizure, can cause rapid blinking or lip smacking, and a few seconds of staring into space. The tonic seizure causes stiffening of the muscles and usually affect muscles in the back, arms and legs. The atonic seizure, also known as drop seizure, cause a loss of muscle control. Clonic seizure is associated with repeated jerking muscle movements affecting the neck, face, and arms. Myoclonic seizures appear as sudden brief jerks or twitches of arms and legs. Tonic-clonic seizures, also known as grand mal seizures, make a person cry out, lose consciousness, fall to the ground or undergo muscle jerks or spasms. Focal seizures are also called partial seizures, possibly because they are located in a single area of the brain. Focal seizures consist of three types of seizures, simple focal seizure, complex focal seizure, and secondary generalized seizure. Simple focal seizures affect a small part of the brain and can cause twitching or a change in sensation, including a change in smell or taste. Complex focal seizures cause confusion or a person to feel dazed. A complex focal seizure may also cause a person to be unable to respond to questions or direction for a few minutes. Secondary generalized seizures begin as a focal seizure located in one side of the brain and develop into a generalized seizure (both sides of the brain).

Diagnosis of epilepsy can include neurological exams and blood tests. Brain abnormalities are detected by electroencephalogram (EEG), high-density EEG, computerized tomography (CT) scan, magnetic resonance imaging (MRI), positron emission tomography (PET) scans, single-photon emission computerized tomography (SPECT), and neuropsychological tests to assess thinking, memory and speech skills. Analysis techniques include statistical parametric mapping (SPM), curry analysis, and magnetoencephalography (MEG).

Treatment with medications (known as antiepileptic or anticonvulsant) or sometimes surgery can control seizures for the majority of people with epilepsy. Some people require lifelong treatment to control seizures, Existing treatments for epilepsy involve medication that can decrease or diminish seizures.

Approximately, 3.4 million people suffer from epilepsy nationwide, including 3 million adults and 470,000 children. (See, Zack M M, et al., National and state estimates of the numbers of adults and children with active epilepsy—United States, 2015. MMWR. 2017; 66:821-825.) In 2015, 1.2% of the total US population were suffering from epilepsy.

### SUMMARY

The present disclosure provides compositions, methods, and dosing regimens for treating epilepsy or seizures in an epileptic patient.

In one aspect, the present disclosure provides a method for treating epilepsy in a subject in need thereof, where the method comprises administering to the subject a pharmaceutically active dose of a therapeutic composition comprising or derived from live non-pathogenic fecal bacteria or a sterile fecal filtrate. In one aspect, a sterile fecal filtrate originates from a donor stool. In another aspect, a sterile fecal filtrate originates from cultured microorganisms.

In another aspect, this disclosure provides use of a composition comprising live non-pathogenic uncultured fecal bacteria in the manufacture of a medication for the treatment of epilepsy.

In one aspect, the present disclosure provides a method for treating epilepsy in a subject in need thereof, where the method comprises administering orally to the subject a pharmaceutically active dose of a therapeutic composition comprising or derived from live, non-pathogenic, synthetic bacterial mixture or live, non-pathogenic, purified or extracted, fecal microbiota, where the dose is administered at a dosing schedule of at least once or twice daily or at least once or twice weekly for at least three, eight, ten, or twenty consecutive weeks. In a further aspect, the dose is administered at a dosing schedule of at least once or twice daily or at least once or twice weekly for at least four, five, six, seven, eleven, twelve, thirteen, fourteen, fifteen, sixteen, seventeen, eighteen, or nineteen consecutive weeks.

In one aspect, the present disclosure provides a method for treating epilepsy in a subject in need thereof, where the method comprises administering orally to the subject a pharmaceutically active dose of a therapeutic composition comprising a liquid, frozen, lyophilized, or encapsulated sterile fecal filtrate, where the dose is administered at a dosing schedule of at least once or twice daily or at least once or twice weekly for at least three, eight, ten, or twenty consecutive weeks.

In one aspect, the present disclosure provides for a method for treating epilepsy or seizure in a subject in need thereof, said method comprising administering to said subject an oral capsule comprising a pharmaceutically active dose of a therapeutic composition comprising live non-pathogenic fecal bacteria or a non-cellular fecal filtrate.

In one aspect, a method achieves a remission, cure, response, or resolution rate of epilepsy of at least about 10%.

In an aspect, a fecal microbiota in a therapeutic composition comprises a donor's substantially entire and non-selected fecal microbiota, reconstituted fecal material, or synthetic fecal material.

In one aspect, the present disclosure provides for a method for treating epilepsy or seizure in a subject in need thereof, the method comprising administering to the subject an oral capsule comprising a pharmaceutically active dose of a therapeutic composition comprising a non-selected fecal microbiota.

In an aspect, the present disclosure provides for a method for treating epilepsy or seizure in a subject in need thereof, the method comprising administering to the subject an oral capsule comprising a pharmaceutically active dose of a therapeutic composition comprising uncultured fecal bacteria.

In one aspect, the present disclosure provides for a method of treating epilepsy or seizure in a subject in need thereof, the method comprising orally administering to the subject a pharmaceutically active dose of a therapeutic composition comprising a non-selected fecal microbiota.

In one aspect, the present disclosure provides for a method of treating epilepsy or seizure in a subject in need thereof, the method comprising determining the relative abundance of one or more Akkermansia or Parabacteroides species in an intestine of the subject and orally administering to the subject a pharmaceutically active dose of a therapeutic composition comprising a non-selected fecal microbiota if the relative abundance of the one or more Akkermansia or Parabacteroides species is less than a threshold level.

In one aspect, the present disclosure provides for a method of treating epilepsy or seizure in a subject in need thereof, the method comprising testing the subject for the relative abundance of one or more Clostridiales, Ruminococcaceae, Rikenellaceae, Lachnospiraceae, and Alistipes species and orally administering to the subject a pharmaceutically active dose of a therapeutic composition comprising a non-selected fecal microbiota.

In one aspect, the present disclosure provides for a method of treating epilepsy or seizure in a subject in need thereof, the method comprising testing the subject for the relative abundance of one or more Proteobacteria, Cronobacter, Bacteroides, Prevotella, and Bifidobacterium species and orally administering to the subject a pharmaceutically active dose of a therapeutic composition comprising a non-selected fecal microbiota.

In one aspect, the present disclosure provides for a method of treating epilepsy or seizure in a subject in need thereof, said method comprising orally administering to the subject a pharmaceutically active dose of a therapeutic composition comprising a non-selected fecal microbiota, wherein the administering comprises at least 10 capsules.

### DETAILED DESCRIPTION

Unless defined otherwise herein, terms are to be understood according to conventional usage by those of ordinary skill in the relevant art.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations of 20%, 10%, 5%, 1%, 0.5%, or even 0.1% of the specified amount.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5%.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, “microbiota,” and “flora” refer to a community of microbes that live in or on a subject's body, both sustainably and transiently, including eukaryotes, archaea, bacteria, and viruses (including bacterial viruses (i.e., phage)). A non-selected fecal microbiota refers to a community or mixture of fecal microbes derived from a donor's fecal sample without selection and substantially resembling microbial constituents and population structure found in such fecal sample.

As used herein, a “sterile fecal filtrate” or a “non-cellular fecal filtrate” refers to a liquid component of a fecal material, where the liquid component is free or substantially free of cell-based living organisms (e.g., bacteria, fungi, or their spores), but retains bacteriophages and non-cellular biological materials. Preferably, a non-cellular or sterile fecal filtrate is also free of viruses for eukaryotic host cells.

As used herein, “remission, cure, or resolution rate” refers to the percentage of patients that are cured or obtain remission or complete resolution of a condition in response to a given treatment. Quantitatively, a patient responds to a treatment positively when the patient's epilepsy is reduced, evident by at least a 10% reduction in the frequency of seizures per day, per week, per month, or per year. Remission, cure, or resolution of epilepsy refers to reduced or no sign of seizures or warnings that a seizure is going to occur.

As used herein, “response rate” refers to the percentage of patients that respond positively (e.g., reduced severity or frequency of one or more symptoms) to a given treatment. A epilepsy patient responds to a treatment positively when the patient shows reduced or no symptoms. A patient also responds positively to a treatment when the patient shows reduced or no seizures.

As used herein, “warning that an epileptic seizure is about to occur”, “warning of an epileptic seizure”, or “sign of an epileptic seizure” refers to a perceptual disturbance which preempts a seizure. For example, warnings or signs of a seizure can include sensitivity to smells, sounds, or sights, anxiety, nausea, dizziness, or visual changes.

As used herein, “eukaryotic” refers to belonging to a cell that contains a nucleus and membrane-bound organelles.

As used herein, “bacteria,” “bacterium,” and “archaea” refer to single-celled prokaryotes that lack membrane bound nuclei and lack organelles.

As used herein, “colony forming units” (cfu) refers to an estimate of the number of viable microorganism cells in a given sample.

As used herein, “viable” means possessing the ability to multiply.

As used herein, “fecal bacteria” refers to bacteria that can be found in fecal matter.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered, for example, daily, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

As used herein, “threshold level” refers to a predetermined level. A threshold level can include the level found in a person without epilepsy. A threshold level can be a level defined to correlate with remission as a result of treatment.

In one aspect, the present disclosure provides for a method for treating a subject in need thereof with a form of seizure selected from the group consisting of generalized and focal seizures. In another aspect, a method provided here is for treating a subject with a generalized seizure selected from the group consisting of absence (petit mal), tonic, atonic, clonic, myoclonic, and tonic-clonic (grand mal) seizures. In a further aspect, a method provided here is for treating a focal seizure selected from the group consisting of simple focal seizure, complex focal seizure, and secondary generalized seizure. In another aspect, a method provided here is for treating a subject in need thereof with a generalized and focal seizures.

In one aspect, the present disclosure provides a method for treating epilepsy in a subject in need thereof, where the method comprises administering to the subject a pharmaceutically active dose of a therapeutic composition comprising live non-pathogenic fecal bacteria. In another aspect, this disclosure provides use of a composition comprising live non-pathogenic fecal bacteria in the manufacture of a medication for the treatment of epilepsy. In one aspect, a therapeutic composition comprises an isolated or purified population of live non-pathogenic fecal bacteria. In one aspect, a therapeutic composition comprises a non-selected fecal microbiota. In another aspect, a therapeutic composition comprises a non-selected and substantially complete fecal microbiota. In another aspect, a therapeutic composition comprises a full-spectrum fecal microbiota.

In an aspect of the present disclosure, a pharmaceutically active dose comprises a pharmaceutical composition comprising a bacterial mixture comprising uncultured fecal bacteria, for example non-selected fecal bacteria or a substantially complete fecal microbiota (e.g., harvested from a healthy human donor). In another aspect of the present disclosure, a pharmaceutically active dose comprises a pharmaceutical composition comprising a bacterial mixture comprising an uncultured collection of fecal bacteria, for example non-selected fecal bacteria or a substantially complete fecal microbiota (e.g., harvested from a healthy human donor). In an aspect, the bacterial mixture further comprises one or more bacterial isolates. In an aspect, the bacterial mixture does not comprise a bacterial isolate.

In one aspect, uncultured fecal bacteria comprises a donor's entire or substantially complete fecal microbiota from a stool sample. In one aspect, uncultured fecal bacteria comprises a non-selective fecal microbiota. In another aspect, uncultured fecal bacteria comprises an isolated or purified population of live non-pathogenic fecal bacteria. In a further aspect, uncultured fecal bacteria comprises a non-selective and substantially complete fecal microbiota preparation from a single donor. In yet another aspect, an uncultured population of fecal bacteria comprises a non-selective and substantially complete fecal microbiota preparation from a single donor. In another aspect, an uncultured collection of fecal bacteria comprises a non-selective and substantially complete fecal microbiota preparation from a single donor. In another aspect, a pharmaceutical composition used herein comprises a mixture of live, non-pathogenic, bacterial isolates and live, non-pathogenic, purified or extracted, uncultured fecal bacteria.

In an aspect, the preparation of uncultured fecal bacteria involves a treatment selected from the group consisting of ethanol treatment, detergent treatment, heat treatment, irradiation, and sonication. In another aspect, the preparation of uncultured fecal bacteria involves no treatment selected from the group consisting of ethanol treatment, detergent treatment, heat treatment, irradiation, and sonication. In one aspect, the preparation of uncultured fecal bacteria involves a separation step selected from the group consisting of density gradients, filtration (e.g., sieves, nylon mesh), and chromatography. In another aspect, the preparation of uncultured fecal bacteria involves no separation step selected from the group consisting of density gradients, filtration (e.g., sieves, nylon mesh), and chromatography. In another aspect, uncultured fecal bacteria comprises an entire or substantially entire fecal microbiota from a stool sample of a subject. In another aspect, a pharmaceutical composition administered herein comprises a fecal microbiota substantially free of donor eukaryotic cells.

In an aspect, a preparation of uncultured fecal bacteria described herein comprises a purified or reconstituted fecal bacterial mixture comprising a mixture 100% similar to a fecal bacterial mixture derived from a donor. In one aspect, a preparation of uncultured fecal bacteria described herein comprises a purified or reconstituted fecal bacterial mixture comprising a mixture 99.9% similar to a fecal bacterial mixture derived from a donor. In another aspect, a preparation of uncultured fecal bacteria described herein comprises a purified or reconstituted fecal bacterial mixture comprising a mixture 98% similar to a fecal bacterial mixture derived from a donor. In another aspect, a preparation of uncultured fecal bacteria described herein comprises a purified or reconstituted fecal bacterial mixture comprising a mixture 97% similar to a fecal bacterial mixture derived from a donor. In a further aspect, a preparation of uncultured fecal bacteria described herein comprises a purified or reconstituted fecal bacterial mixture comprising a mixture 96% similar to a fecal bacterial mixture derived from a donor. In yet another aspect, a preparation of uncultured fecal bacteria described herein comprises a purified or reconstituted fecal bacterial mixture comprising a mixture 95, 94, 93, 92, 91, 90, 89, 88, 87, 85, 84, 83, 82, 81, 80, 75, 70, 65, 60, 55, 50, 45, or 40% similar to a fecal bacterial mixture derived from a donor. In another aspect, a preparation of uncultured fecal bacteria described herein comprises a purified or reconstituted fecal bacterial mixture comprising a mixture that is between 100 and 95, 95 and 90, 90 and 85, 85 and 80, 80 and 75, 75 and 70, 70 and 65, and 65 and 60% similar to a fecal bacterial mixture derived from a donor.

In an aspect, a pharmaceutically active dose provided or administered herein comprises a pharmaceutical composition comprising uncultured fecal bacteria comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a pharmaceutical composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a pharmaceutical composition comprises a preparation of flora in proportional content that resembles a normal healthy human fecal flora.

In aspects of the present disclosure, a pharmaceutically active dose comprises a pharmaceutical composition comprising a bacterial mixture comprising uncultured fecal bacteria, for example non-selected fecal bacteria. In an aspect, a bacterial mixture comprises a single bacterial isolate or multiple bacterial isolates (e.g., in the form of a bacterial cocktail). In an aspect, a pharmaceutical composition comprises a bacterial mixture comprising (i) an uncultured population of fecal bacteria; and (ii) at least one bacterial isolate. In another aspect, a pharmaceutical composition comprises a bacterial mixture comprising (i) uncultured fecal bacteria; and (ii) at least one bacterial isolate. Such a bacterial mixture can be referred to as uncultured fecal bacteria enriched, supplemented or “spiked” with one or more bacterial isolates. By enriching or spiking a population of uncultured bacteria derived from a stool sample (e.g., a fecal microbiota) of a healthy donor with one or more non-pathogenic bacterial isolates, a composition can be produced in which the amount of a particular bacterial strain or strains (i.e. the spiked-in bacterial isolate(s)) can be accounted for and precisely controlled. Without being bound by theory, this is advantageous, for example, where the at least one bacterial isolate spiked into the uncultured fecal bacteria is important for or involved in the treatment of a subject (e.g., having or susceptible to acquiring one or more symptoms of epilepsy), but insufficient on its own to generate an enhanced or optimal treatment response in the subject. Probiotics are relied on for their effect associated with the administration of a single bacterial isolate or a few bacterial isolates. Unlike probiotics, administration to an epileptic subject of one or more bacterial isolates together with uncultured fecal bacteria (i.e., derived from a healthy donor) provides the subject with the advantage of the administered bacterial isolate combined with multi-factorial benefits conferred by the additional fecal bacterial strains present in the uncultured population. These additional fecal bacterial strains may combine to, for example, provide for the necessary context or interactions (e.g. via one or more released factors) to enable the bacterial isolate to induce an optimal response in the subject, or may directly induce a response in the subject that combines and/or synergizes with a response induced by the bacterial isolate to treat the subject. Accordingly, in certain aspects, a pharmaceutical composition comprising a mixture of one or more bacterial isolates and uncultured fecal bacteria can be more effective in treating a subject (e.g., having or susceptible to acquiring one or more symptoms of Epilepsy) than a composition comprising the bacterial isolate alone.

In an aspect, a method further comprises administering a narrow-spectrum antiepileptic medication. Narrow-spectrum antiepileptic medication are utilized if seizures are isolated to a specific part of the brain. In another aspect, a narrow-spectrum antiepileptic medication is selected from the group consisting of carbamazepine, benzodiazepines including clobazam, diazepam, divalproex, eslicarbazepine acetate, ethosuximide, gabapentin, lacosamide, methsuximide, oxcarbazepine, perampanel, phenobarbital, phenytoin, pregabalin, refinamide, tiagabine hydrochloride, vigabatril, and a combination thereof. In another aspect, a method further comprises administering a broad-spectrum antiepileptic medication. Broad-spectrum antiepileptic medication is utilized to treat seizures which affect multiple sections of the brain. In another aspect, a broad-spectrum antiepileptic medication is selected from the group consisting of clonazepam, clorazepate, ezogabine, felbamate, lamotrigine, levetiracetam, lorazepam, primidone, topiramate, valproic acid, zonisamide and a combination thereof.

In another aspect, a method further comprises administering a benzodiazepines. In yet another aspect, a method also comprises administering diazepam, clonazepam or lorazepam. In a further aspect, a method also comprises administering medication selected from the group consisting of Acetazolamide, carbamazepine, clobazam, clonazepam, diazepam, ethosuximide, Fosphenytoin, gabapentin, lacosamide, lamotrigine, levetiracetam, lorazepam, methsuximide, nitrazepam, oxcarbazepine, paraldehyde, phenobarbital, phenobarb, phenobarbital sodium, phenytoin, pregabalin, primidone, refinamide, sodium valproate, stiripentol, topiramate, valproic acid, vigabatrin, felbamate, tiagabine hydrochloride, and zonisamide.

In an aspect, the present disclosure provides a method for treating epilepsy in a subject in need thereof, where the method comprising administering to the subject a pharmaceutically active dose of a therapeutic composition comprising live non-pathogenic bacteria. In another aspect the subject is not co-administered a narrow-spectrum antiepileptic medication. In another aspect, the subject is not co-administered a broad-spectrum antiepileptic medication. In yet another aspect, the subject is not administered an antiepileptic medication at least 1, 2, 3, 4, 5, 6, 10, 15, 20, 25, 30, 35, 40, 45, or 50 weeks prior to the administering of a pharmaceutically active dose of a therapeutic composition comprising live non-pathogenic bacteria. In a further aspect, the subject is not administered an antiepileptic medication at least 1, 2, 3, 4, 5, 6, 7, 7, 9, or 10 years prior to the administering of a pharmaceutically active dose of a therapeutic composition comprising live non-pathogenic bacteria. In another aspect, the subject has not been administered an antiepileptic medication prior to the administering of a pharmaceutically active dose of a therapeutic composition comprising live non-pathogenic bacteria.

In an aspect, the present disclosure provides a method for treating epilepsy in a subject in need thereof, where the method comprises administering to the subject a pharmaceutically active dose of a therapeutic composition comprising live non-pathogenic bacteria. In one aspect, the present disclosure provides a method for treating epilepsy in a subject in need thereof, where the method comprises administering daily to the subject a pharmaceutically active dose of a therapeutic composition comprising live non-pathogenic fecal bacteria. In one aspect, a therapeutic composition is administered to an epileptic patient in need thereof at least once daily or weekly for at least two consecutive days or weeks. In one aspect, a therapeutic composition is administered at least once daily or weekly for at least 3, 4, 5, 6, 7, 8, 9, 10, 11, 12, 13, 14, or 15 consecutive days or weeks. In another aspect, a therapeutic composition is administered at least once daily or weekly for at least 1, 2, 3, 4, 5, 6, 7, 8, 9, 10, 11, or 12 consecutive days or weeks. In one aspect, a therapeutic composition is administered at least once daily or weekly for at most 4, 5, 6, 7, 8, 9, 10, 11, 12, 13, 14, 15, 16, 17, 18, 19, or 20 consecutive days or weeks. In another aspect, a therapeutic composition is administered at least once daily or weekly for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an epileptic patient in need thereof at least twice daily or weekly for at least two consecutive days or weeks. In one aspect, a therapeutic composition is administered at least twice daily or weekly for at least 3, 4, 5, 6, 7, 8, 9, 10, 11, 12, 13, 14, or 15 consecutive days or weeks. In another aspect, a therapeutic composition is administered at least twice daily or weekly for at least 1, 2, 3, 4, 5, 6, 7, 8, 9, 10, 11, or 12 consecutive days or weeks. In one aspect, a therapeutic composition is administered at least twice daily or weekly for at most 4, 5, 6, 7, 8, 9, 10, 11, 12, 13, 14, 15, 16, 17, 18, 19, or 20 consecutive days or week. In another aspect, a therapeutic composition is administered at least twice daily or weekly for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an epileptic patient in need thereof at least three times daily or weekly for at least two consecutive days or weeks. In one aspect, a therapeutic composition is administered at least three times daily or weekly for at least 3, 4, 5, 6, 7, 8, 9, 10, 11, 12, 13, 14, or 15 consecutive days or weeks. In another aspect, a therapeutic composition is administered at least three times daily or weekly for at least 1, 2, 3, 4, 5, 6, 7, 8, 9, 10, 11, or 12 consecutive days or weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating epilepsy in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily or weekly for at least three consecutive days or weeks. In another aspect, a dose is administered at least once, twice, or three times daily or weekly for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating epilepsy in a subject in need thereof, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, a subject being treated is a subject already with epilepsy. Administration of a disclosed therapeutic composition to clinically, asymptomatic human subject who is genetically predisposed or prone to epilepsy is also useful in preventing the onset of clinical symptoms of epilepsy. A human subject genetically predisposed or prone to epilepsy can be a human subject having a close family member or relative exhibiting or having suffered epilepsy. In another aspect, a subject being treated is a subject in which epilepsy is to be prevented. In another aspect, a subject being treated is predisposed or susceptible to multiples sclerosis. In another aspect, a subject being treated is a subject diagnosed as having epilepsy. In one aspect, a subject being treated is a patient in need thereof. In another aspect, a patient being treated is immunocompromised. In another aspect, a patient is a patient with epilepsy but otherwise healthy. In another aspect, a patient is of healthy nutrition. In yet another aspect, a patient has normal growth. In another aspect, a patient has a healthy, balanced microbiota. In another aspect, a patient does not have dysbiosis at the time of treatment (e.g., Ulcerative Colitis, Crohn's Disease, Irritable Bowel Syndrome, etc.). In another aspect, a patient is an epileptic patient in remission. In another aspect, a patient is an epileptic patient in remission as a result of treatment. In yet another aspect, a patient has no prior history of seizures or epilepsy. In a further aspect, a patient has experienced at most 1, 2, or 3 seizures in their lifetime. In another aspect, a patient has experienced at most 5 or 10 seizures in their lifetime. In another aspect, a patient is a post-traumatic seizure patient. In another aspect, a patient is a person at risk of seizures. In yet another aspect, a person at risk of seizures is a person who has experienced a head trauma. In another aspect, a person at risk of seizures is a person with inflammation of the brain. In another aspect, a person at risk of seizures is a person with a brain infection. In another aspect, a person at risk of seizures is a person who has suffered a stroke. In another aspect, a person at risk of seizures is a person with a family member with epilepsy or seizures.

In one aspect, a subject being treated is a human patient. In one aspect, a patient is a male patient. In one aspect, a patient is a female patient. In one aspect, a patient is a premature newborn. In an aspect, a patient is a male premature newborn. In another aspect, a patient is a female premature newborn. In one aspect, a patient is a term newborn. In an aspect, a patient is a male term newborn. In another aspect, a patient is a female term newborn. In one aspect, a patient is a neonate. In one aspect, a patient is an infant. In another aspect, a patient is a male infant. In another aspect, a patient is a female infant. In one aspect, a patient is a toddler. In another aspect, a patient is a male toddler. In another aspect, a patient is a female toddler. In one aspect, a patient is a young child. In one aspect, a patient is a child. In another aspect, a patient is a male child. In another aspect, a patient is a female child. In one aspect, a patient is an adolescent. In one aspect, a patient is a pediatric patient. In another aspect, a patient is a male pediatric patient. In another aspect, a patient is a female pediatric patient. In one aspect, a patient is a geriatric patient. In another aspect, a patient is a male geriatric patient. In another aspect, a patient is a female geriatric patient. In one aspect, a patient is an adult male. In another aspect, the patient is an adult female. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a subject being treated is a patient on a limited diet. In another aspect, a subject being treated is a patient on a non-limited diet. In another aspect, a subject being treated is a patient on a diet comprising animal protein. In another aspect, a subject being treated is a patient on a diet comprising spicy foods. In another aspect, a subject being treated is a patient on a diet comprising high fat food.

In one aspect, a method comprises administering a therapeutic composition orally, by enema, or via rectal suppository. In one aspect, a therapeutic composition administered herein is formulated as an enteric coated (and/or acid-resistant) capsule or microcapsule, or formulated as part of or administered together with a food, a food additive, a dairy-based product, a soy-based product or a derivative thereof, a jelly, flavored liquid, ice block, ice cream, or a yogurt. In another aspect, a therapeutic composition administered herein is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is homogenized, lyophilized, pulverized and powdered. It may then be infused, dissolved such as in saline, as an enema. Alternatively the powder may be encapsulated as enteric-coated and/or acid-resistant delayed release capsules for oral administration. In an aspect, the powder may be double encapsulated with acid-resistant/delayed release capsules for oral administration. These capsules may take the form of enteric-coated and/or acid-resistant delayed release microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, spray-dried, foam-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a pharmaceutical composition comprises a lyophilized formulation further comprising an antioxidant. In another aspect, a pharmaceutical composition comprises a lyophilized formulation further comprising an antioxidant and a reducing agent. In yet another aspect, a pharmaceutical composition comprises a lyophilized formulation further comprising a reducing agent. In certain embodiments, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, Glutathione, Methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a therapeutic composition comprises a cryoprotectant. As used herein, a “cryoprotectant” refers to a substance that is added to a formulation in order to protect an active ingredient during freezing. In an aspect, a cryoprotectant comprises, consists essentially of, or consists of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof. In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehaolse with 0.1% Polysorbate 80.

In another aspect, a therapeutic composition comprises a lyoprotectant. As used herein, a “lyoprotectant” refers to a substance that is added to a formulation in order to protect an active ingredient during the drying stage of a lyophilization (also known as freeze-drying) process. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consists essentially of, or consists of, one or more substances mentioned in this paragraph and the paragraph above.

In one aspect, a cryoprotectant or a lyoprotectant comprise an intracellular agent, e.g., DMSO, Glycerol, or PEG, which penetrates inside the cell preventing the formation of ice crystals that could result in membrane rupture. In another aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material. In another aspect, a therapeutic composition administered does not comprise an acid suppressant, an antacid, an H2 antagonist, a proton pump inhibitor or a combination thereof. In yet another aspect, a therapeutic composition administered does not comprise an acid suppressant. In another aspect, a therapeutic composition administered does not comprise an antacid. In another aspect, a therapeutic composition administered does not comprise an H2 antagonist. In another aspect, a therapeutic composition administered does not comprise a proton pump inhibitor. In another aspect, a therapeutic composition administered does not comprise metoclopramide.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides. In another aspect, a subject is not pretreated with a prebiotic nutrient prior to treatment with a therapeutic composition. In another aspect, the therapeutic composition is not supplemented with a prebiotic nutrient.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a therapeutic bacterial or microbiota composition. In another aspect, the therapeutic composition is not supplemented with an antibiotic composition. In a further aspect, a method further comprises pretreating a subject with an anti-inflammatory drug prior to administration of a therapeutic bacterial or microbiota composition. In yet another aspect, a subject is not pretreated with an anti-inflammatory drug prior to administering a therapeutic bacterial or microbiota composition. In another aspect, a therapeutic bacterial or microbiota composition is not supplemented with an anti-inflammatory.

In an aspect of the present disclosure, a method further comprises administering a therapeutic bacterial or microbiota composition to a subject in need thereof, without co-administering steroids. In another aspect, the subject has not been previously treated with steroids to treat a dysbiosis. In yet another aspect, the subject is not administered a steroid at least 1, 2, 3, 4, 5, 6, 10, 15, 20, 25, 30, 35, 40, 45, or 50 weeks prior to the administering of a therapeutic composition. In a further aspect, the subject is not administered a steroid at least 1, 2, 3, 4, 5, 6, 7, 7, 9, or 10 years prior to the administering of a therapeutic composition. In yet another aspect, the subject is not treated with steroids for at least 1, 2, 3, or 4 weeks prior to or after the administering of a therapeutic composition comprising fecal microbiota. In another aspect, the subject is not co-treated with drugs to treat conditions of dysbiosis (e.g., Crohn's disease, Ulcerative Colitis, Irritable Bowel Disease, etc.). In yet another aspect, a subject is not co-treated with thiopurines or 5-aminoscalicylate (5-ASA). In a further aspect, a subject is not co-treated with a corticosteroid, 5-ASA products, immunomodulators, anti-TNFα agents, or other medication prescribed to treat Crohn's disease, Ulcerative Colitis, Irritable Bowel Syndrome, and Irritable Bowel Disease. In another aspect, a subject is not co-treated with a drug used to treat gastrointestinal disorders.

In an aspect of the present disclosure, a method further comprises administering a therapeutic bacterial or microbiota composition to a subject in need thereof, without co-administering nonsteroidal anti-inflammatory drugs. In another aspect, the subject has not been previously treated with nonsteroidal anti-inflammatory drugs to prevent ulcerative colitis flare-ups. In yet another aspect, the subject is not administered a nonsteroidal anti-inflammatory drug at least 1, 2, 3, 4, 5, 6, 10, 15, 20, 25, 30, 35, 40, 45, or 50 weeks prior to the administering of a therapeutic composition. In a further aspect, the subject is not administered a nonsteroidal anti-inflammatory drug at least 1, 2, 3, 4, 5, 6, 7, 7, 9, or 10 years prior to the administering of a therapeutic composition. In yet another aspect, the subject is not treated with nonsteroidal anti-inflammatory drug for at least 1, 2, 3, or 4 weeks prior to or after the administering of a therapeutic composition comprising fecal microbiota. In another aspect, the subject is not treated with mesalamine for at least 1, 2, 3, or 4 weeks prior to or after the administering of a therapeutic composition comprising fecal microbiota.

In one aspect, a method achieves a remission, cure, response, or resolution rate of seizures of at least about 10%, 15%, 20%, 25%, 30%, 35%, 40%, 45%, 50%, 55%, 60%, 65%, 70%, 75%, 80%, 85%, 90%, 95%, 97%, or 99%. In one aspect, a treatment method achieves at least 10%, 20%, 30%, 50%, 60%, 70%, 80%, or 90% reduction of seizures after 4, 8, or 12 weeks of treatment compared to baseline (e.g., immediately prior to treatment). In one aspect, a treatment method achieves at least 10%, 20%, 30%, 50%, 60%, 70%, 80%, or 90% reduction of frequency of seizures in at least 10%, 20%, 30%, 50%, 60%, 70%, 80%, or 90% patients after 4, 8, or 12 weeks of treatment compared to baseline (e g, immediately prior to treatment). In an aspect, the present disclosure provides a method which achieves a 10%, 20%, 30%, 40%, 50%, 60%, 70%, 80%, or 90% reduction of a patient's frequency of seizures after 1, 2, 3, 4, 5, 6, 7, 8, 9, 10, 11, or 12 weeks or months of treatment. In another aspect, a method achieves a 10-20%, 20-30%, 30-40%, 40-50%, 50-60%, 60-70%, 70-80%, 80-90%, or 90-99% reduction of a patient's frequency of seizures after 1, 2, 3, 4, 5, 6, 7, 8, 9, 10, 11, or 12 weeks or months of treatment.

In one aspect, a method achieves a remission, cure, response, or resolution rate of epilepsy of between about 10-20%, 20-30%, 30-40%, 40-50%, 50-60%, 60-70%, 70-80%, 80-90%, or 90-99%. In one aspect, a treatment method achieves between about 10-20%, 20-30%, 30-40%, 40-50%, 50-60%, 60-70%, 70-80%, 80-90%, or 90-99% reduction of epilepsy disease symptoms after 4, 8, or 12 weeks of treatment compared to baseline (e.g., immediately prior to treatment). In one aspect, a treatment method achieves between about 10-20%, 20-30%, 30-40%, 40-50%, 50-60%, 60-70%, 70-80%, 80-90%, or 90-99% reduction of epilepsy disease symptoms in about 10-20%, 20-30%, 30-40%, 40-50%, 50-60%, 60-70%, 70-80%, 80-90%, or 90-99% of patients after 4, 8, or 12 weeks of treatment compared to baseline (e.g., immediately prior to treatment). In one aspect, a treatment method achieves between about 10-20%, 20-30%, 30-40%, 40-50%, 50-60%, 60-70%, 70-80%, 80-90%, or 90-99% reduction of epilepsy disease symptoms after 2, 4, 6, 8, 10, 12 or 14 days of treatment compared to baseline (e.g., immediately prior to treatment). In one aspect, a treatment method achieves between about 10-20%, 20-30%, 30-40%, 40-50%, 50-60%, 60-70%, 70-80%, 80-90%, or 90-99% reduction of epilepsy disease symptoms in about 10-20%, 20-30%, 30-40%, 40-50%, 50-60%, 60-70%, 70-80%, 80-90%, or 90-99% of patients after 2, 4, 6, 8, 10, 12 or 14 days of treatment compared to baseline (e g, immediately prior to treatment).

In an aspect of the present disclosure, an epileptic patient can receive a warning that a seizure is about to occur. In one aspect, a treatment method achieves at least 10%, 20%, 30%, 50%, 60%, 70%, 80%, or 90% reduction of the frequency of warnings or signs that an epileptic seizure is about to occur after 4, 8, or 12 weeks of treatment compared to baseline (e.g., immediately prior to treatment). In one aspect, a treatment method achieves at least 10%, 20%, 30%, 50%, 60%, 70%, 80%, or 90% reduction of the frequency of warnings or signs that an epileptic seizure is about to occur in at least 10%, 20%, 30%, 50%, 60%, 70%, 80%, or 90% patients after 4, 8, or 12 weeks of treatment compared to baseline (e.g., immediately prior to treatment).

In one aspect, a treatment method achieves between about 10-20%, 20-30%, 30-40%, 40-50%, 50-60%, 60-70%, 70-80%, 80-90%, or 90-99% reduction of the frequency of warnings that an epileptic seizure is about to occur after 4, 8, or 12 weeks of treatment compared to baseline (e.g., immediately prior to treatment). In one aspect, a treatment method achieves between about 10-20%, 20-30%, 30-40%, 40-50%, 50-60%, 60-70%, 70-80%, 80-90%, or 90-99% reduction of the frequency of warnings that an epileptic seizure is about to occur in between about 10-20%, 20-30%, 30-40%, 40-50%, 50-60%, 60-70%, 70-80%, 80-90%, or 90-99% of patients after 4, 8, or 12 weeks of treatment compared to baseline (e.g., immediately prior to treatment).

In an aspect the present disclosure provides for a method that prevents relapse of seizures. In an aspect, the method prevents relapse of seizure during treatment for at least 1, 2, 3, 4, 5, 6, 7, 8, 9, 10, 11, 12, 13, 14, 15, 20, 25, 30, 35, 40, 45, 50, or 55 days or weeks from the start of treatment. In another aspect, the method prevents relapse of seizure during treatment for between land 2, 2 and 4, 4 and 6, 6 and 8, 8 and 10, 10 and 20, 20 and 30, 30 and 40, or 40 and 50 days or weeks from the start of treatment. In another aspect, the method prevents relapse of seizure during after treatment has stopped for at least 1, 2, 3, 4, 5, 6, 7, 8, 9, 10, 11, 12, 13, 14, 15, 20, 25, 30, 35, 40, 45, 50, or 55 days or weeks after the last treatment. In another aspect, the method prevents relapse of seizure after treatment has stopped for between land 2, 2 and 4, 4 and 6, 6 and 8, 8 and 10, 10 and 20, 20 and 30, 30 and 40, or 40 and 50 days or weeks after the last treatment.

In an aspect the present disclosure provides for a method comprising orally administering to a subject a pharmaceutically active dose of a therapeutic composition comprising live non-pathogenic fecal bacteria or a non-cellular fecal filtrate. In one aspect, the subject is deficient in one or more of Akkermansia or Parabacteroides species relative to a subject who does not have epilepsy. In another aspect, the subject in need thereof is non-responsive to ketogenic diet (KD) therapy. In another aspect, the subject in need thereof is pretreated with a probiotic prior to administration of said composition. In a further aspect, the subject in need thereof is simultaneously treated with a probiotic. In an even further aspect, the probiotic is selected from the group consisting of Akkermansia or Parabacteroides species. In another aspect, the probiotic is one or more Akkermansia or Parabacteroides species.

In an aspect, the present disclosure provides for a method comprising testing a subject for the relative abundance of one or more Akkermansia or Parabacteroides species and orally administering to the subject a pharmaceutically active dose of a therapeutic composition comprising live non-pathogenic fecal bacteria.

In an aspect, the present disclosure provides for a method comprising testing a subject for the relative abundance of one or more Clostridiales, Ruminococcaceae, Rikenellaceae, Lachnospiraceae, and Alistipes species and orally administering to said subject a pharmaceutically active dose of a therapeutic composition comprising live non-pathogenic fecal bacteria.

In an aspect, the present disclosure provides for a method comprising determining the relative abundance of one or more Proteobacteria, Cronobacter, Bacteroides, Prevotella, and Bifidobacterium species in an intestine of the subject and orally administering to the subject a pharmaceutically active dose of a therapeutic composition comprising live non-pathogenic fecal bacteria.

In an aspect, the present disclosure provides for a method of diagnosing epilepsy in a subject in need thereof comprising collecting stool from the subject and subjecting the stool to HPLC.

In another aspect, the present disclosure provides for a method of treating epilepsy or seizure in a subject in need thereof, the method comprising orally administering to the subject a pharmaceutically active dose of a therapeutic composition comprising a non-selected fecal microbiota. In another aspect, the method comprises administering to the subject an oral capsule comprising a pharmaceutically active dose of a therapeutic composition comprising a non-selected fecal microbiota.

In another aspect, the present disclosure provides for a method of treating epilepsy or seizure in a subject in need thereof, the method comprising determining the relative abundance of one or more Akkermansia or Parabacteroides species in an intestine of the subject and orally administering to the subject a pharmaceutically active dose of a therapeutic composition comprising a non-selected fecal microbiota if the relative abundance of the one or more Akkermansia or Parabacteroides species is less than a threshold level. In another aspect, the method comprises testing the relative abundance of two or more Akkermansia or Parabacteroides species. In yet another aspect, the method comprises testing the relative abundance of three or more Akkermansia or Parabacteroides species. In another aspect, the method comprises testing the relative abundance of four or more Akkermansia or Parabacteroides species. In a further aspect, the method comprises testing the relative abundance of five or more Akkermansia or Parabacteroides species. In another aspect, the method comprises testing the relative abundance of six or more Akkermansia or Parabacteroides species. In another aspect, the method comprises testing the relative abundance of at least one Akkermansia or Parabacteroides species. In yet another aspect, the method comprises testing the relative abundance of at least three Akkermansia or Parabacteroides species. In another aspect, the method comprises testing the relative abundance of at least five Akkermansia or Parabacteroides species. In a further aspect, the method comprises testing the relative abundance of at least eight Akkermansia or Parabacteroides species. In another aspect, the method comprises testing the relative abundance of six or more Akkermansia or Parabacteroides species.

In a further aspect, the subject in need thereof is deficient in one or more of Akkermansia or Parabacteroides species relative to a subject who does not have epilepsy. In another aspect, the subject in need thereof is deficient in two or more of Akkermansia or Parabacteroides species relative to a subject who does not have epilepsy. In another aspect, the subject in need thereof is deficient in three or more of Akkermansia or Parabacteroides species relative to a subject who does not have epilepsy. In yet another aspect, the subject in need thereof is deficient in four or more of Akkermansia or Parabacteroides species relative to a subject who does not have epilepsy. In a further aspect, the subject in need thereof is deficient in five or more of Akkermansia or Parabacteroides species relative to a subject who does not have epilepsy. In another aspect, the subject in need thereof is deficient in 1 to 3, 3 to 5, or 5 to 10 Akkermansia or Parabacteroides species relative to a subject who does not have epilepsy.

In one aspect, the present disclosure provides for a method of treating epilepsy or seizure in a subject in need thereof, the method comprising testing the subject for the relative abundance of one or more Clostridiales, Ruminococcaceae, Rikenellaceae, Lachnospiraceae, and Alistipes species and orally administering to the subject a pharmaceutically active dose of a therapeutic composition comprising a non-selected fecal microbiota. In another aspect, the method comprises testing the relative abundance of two or more Clostridiales, Ruminococcaceae, Rikenellaceae, Lachnospiraceae, and Alistipes species. In yet another aspect, the method comprises testing the relative abundance of three or more Clostridiales, Ruminococcaceae, Rikenellaceae, Lachnospiraceae, and Alistipes species. In another aspect, the method comprises testing the relative abundance of four or more Clostridiales, Ruminococcaceae, Rikenellaceae, Lachnospiraceae, and Alistipes species. In a further aspect, the method comprises testing the relative abundance of five or more Clostridiales, Ruminococcaceae, Rikenellaceae, Lachnospiraceae, and Alistipes species. In another aspect, the method comprises testing the relative abundance of six or more Clostridiales, Ruminococcaceae, Rikenellaceae, Lachnospiraceae, and Alistipes species. In another aspect, the method comprises testing the relative abundance of at least one Clostridiales, Ruminococcaceae, Rikenellaceae, Lachnospiraceae, and Alistipes species. In yet another aspect, the method comprises testing the relative abundance of at least three Clostridiales, Ruminococcaceae, Rikenellaceae, Lachnospiraceae, and Alistipes species. In another aspect, the method comprises testing the relative abundance of at least five Clostridiales, Ruminococcaceae, Rikenellaceae, Lachnospiraceae, and Alistipes species. In a further aspect, the method comprises testing the relative abundance of at least eight Clostridiales, Ruminococcaceae, Rikenellaceae, Lachnospiraceae, and Alistipes species. In another aspect, the method comprises testing the relative abundance of six or more Clostridiales, Ruminococcaceae, Rikenellaceae, Lachnospiraceae, and Alistipes species.

In one aspect, the subject in need thereof is deficient in one or more Clostridiales, Ruminococcaceae, Rikenellaceae, Lachnospiraceae, and Alistipes species. In another aspect, the subject in need thereof is deficient in two or more Clostridiales, Ruminococcaceae, Rikenellaceae, Lachnospiraceae, and Alistipes species. In another aspect, the subject in need thereof is deficient in three or more Clostridiales, Ruminococcaceae, Rikenellaceae, Lachnospiraceae, and Alistipes species. In yet another aspect, the subject in need thereof is deficient in four or more Clostridiales, Ruminococcaceae, Rikenellaceae, Lachnospiraceae, and Alistipes species. In a further aspect, the subject in need thereof is deficient in five or more Clostridiales, Ruminococcaceae, Rikenellaceae, Lachnospiraceae, and Alistipes species.

In one aspect, the present disclosure provides for a method of treating epilepsy or seizure in a subject in need thereof, the method comprising testing the subject for the relative abundance of one or more Proteobacteria, Cronobacter, Bacteroides, Prevotella, and Bifidobacterium species and orally administering to the subject a pharmaceutically active dose of a therapeutic composition comprising a non-selected fecal microbiota. In another aspect, the method comprises testing the relative abundance of two or more Proteobacteria, Cronobacter, Bacteroides, Prevotella, and Bifidobacterium species. In yet another aspect, the method comprises testing the relative abundance of three or more Proteobacteria, Cronobacter, Bacteroides, Prevotella, and Bifidobacterium species. In another aspect, the method comprises testing the relative abundance of four or more Proteobacteria, Cronobacter, Bacteroides, Prevotella, and Bifidobacterium species. In a further aspect, the method comprises testing the relative abundance of five or more Proteobacteria, Cronobacter, Bacteroides, Prevotella, and Bifidobacterium species. In another aspect, the method comprises testing the relative abundance of six or more Proteobacteria, Cronobacter, Bacteroides, Prevotella, and Bifidobacterium species. In another aspect, the method comprises testing the relative abundance of at least one Proteobacteria, Cronobacter, Bacteroides, Prevotella, and Bifidobacterium species. In yet another aspect, the method comprises testing the relative abundance of at least three Proteobacteria, Cronobacter, Bacteroides, Prevotella, and Bifidobacterium species. In another aspect, the method comprises testing the relative abundance of at least five Proteobacteria, Cronobacter, Bacteroides, Prevotella, and Bifidobacterium species. In a further aspect, the method comprises testing the relative abundance of at least eight Proteobacteria, Cronobacter, Bacteroides, Prevotella, and Bifidobacterium species. In another aspect, the method comprises testing the relative abundance of six or more Proteobacteria, Cronobacter, Bacteroides, Prevotella, and Bifidobacterium species.

In one aspect, the subject in need thereof is deficient in one or more Proteobacteria, Cronobacter, Bacteroides, Prevotella, and Bifidobacterium species. In another aspect, the subject in need thereof is deficient in two or more Proteobacteria, Cronobacter, Bacteroides, Prevotella, and Bifidobacterium species. In another aspect, the subject in need thereof is deficient in three or more Proteobacteria, Cronobacter, Bacteroides, Prevotella, and Bifidobacterium species. In yet another aspect, the subject in need thereof is deficient in four or more Proteobacteria, Cronobacter, Bacteroides, Prevotella, and Bifidobacterium species. In a further aspect, the subject in need thereof is deficient in five or more Proteobacteria, Cronobacter, Bacteroides, Prevotella, and Bifidobacterium species.

In one aspect, the present disclosure provides for a method of treating epilepsy or seizure in a subject in need thereof, said method comprising orally administering to the subject a pharmaceutically active dose of a therapeutic composition comprising a non-selected fecal microbiota, wherein the administering comprises at least 10 capsules.

In one aspect, the present disclosure provides for a method of treating epilepsy or seizure in a subject in need thereof, comprises pretreating or co-administering the subject with one or more medications selected from the group consisting of carbamazepine, clobazam, diazepam, divalproex, eslicarbazepine acetate, ethosuximide, gabapentin, lacosamide, methsuximide, oxcarbazepine, perampanel, phenobarbital, phenytoin, pregabalin, refinamide, tiagabine hydrochloride, vigabatril, and a combination thereof. In a further aspect, the method comprises pretreating or co-administering the subject with two or more medications selected from the group consisting of carbamazepine, clobazam, diazepam, divalproex, eslicarbazepine acetate, ethosuximide, gabapentin, lacosamide, methsuximide, oxcarbazepine, perampanel, phenobarbital, phenytoin, pregabalin, refinamide, tiagabine hydrochloride, vigabatril, and a combination thereof.

In another aspect, the method comprises pretreating or co-administering one or more medications selected from the group consisting of clonazepam, clorazepate, ezogabine, felbamate, lamotrigine, levetiracetam, lorazepam, primidone, topiramate, valproic acid, zonisamide and a combination thereof. In yet another aspect, the method comprises pretreating or co-administering two or more medications selected from the group consisting of clonazepam, clorazepate, ezogabine, felbamate, lamotrigine, levetiracetam, lorazepam, primidone, topiramate, valproic acid, zonisamide and a combination thereof.

In an aspect, the present disclosure provides for administering one or more antiepileptic medications prior to administering a pharmaceutically active dose of a therapeutic composition provided in the disclosure. In another aspect, the one or more antiepileptic medication is continued during the administering of a pharmaceutically active dose of a therapeutic composition provided in the disclosure. In another aspect, the one or more antiepileptic medication is discontinued during the administering of a pharmaceutically active dose of a therapeutic composition provided in the disclosure. In yet another aspect, the one or more antiepileptic medication is administered prior to and after the completion of the administering of a pharmaceutically active dose of a therapeutic composition provided in the disclosure. In a further aspect, the administering of one or more antiepileptic medication is decreased in dose during the administering of a pharmaceutically active dose of a therapeutic composition provided in the disclosure, compared to the dose prior to the administering of a pharmaceutically active dose of a therapeutic composition. In yet another aspect, the dose of the one or more antiepileptic medication is decreased after the administering of a pharmaceutically active dose of a therapeutic composition provided in the disclosure, compared to the dose prior to the administering of a pharmaceutically active dose of a therapeutic composition. In yet another aspect, the dose of the one or more antiepileptic medication is increased after the administering of a pharmaceutically active dose of a therapeutic composition provided in the disclosure, compared to the dose prior to the administering of a pharmaceutically active dose of a therapeutic composition. In a further aspect, the dose of the one or more antiepileptic medication is decreased in dose after the administering of a pharmaceutically active dose of a therapeutic composition provided in the disclosure, compared to the dose of the one or more antiepileptic medication during the administering of a pharmaceutically active dose of a therapeutic composition.

In an aspect, the present disclosure provides for administering a probiotic prior to administering a therapeutic composition comprising a non-selected fecal microbiota. In another aspect, the disclosure provides for co-administering a probiotic and a therapeutic composition comprising a non-selected fecal microbiota. In another aspect, the probiotic is selected from the group consisting of one or more Akkermansia or Parabacteroides species. In another aspect, the probiotic is selected from the group consisting of two or more Akkermansia or Parabacteroides species. In another aspect, the probiotic is selected from the group consisting of three or more Akkermansia or Parabacteroides species. In another aspect, the probiotic is selected from the group consisting of four or more Akkermansia or Parabacteroides species. In another aspect, the probiotic is selected from the group consisting of five or more Akkermansia or Parabacteroides species. In another aspect, the probiotic is selected from the group consisting of six or more Akkermansia or Parabacteroides species. In another aspect, the probiotic is selected from the group consisting of 1 to 3, 3 to 5, 5 to 8, or 8 to 10 Akkermansia or Parabacteroides species. A probiotic may be provided in a single dose or in multiple doses. When provided as a single composition, the single composition may comprise a single probiotic or a mixture of probiotics. When provided in multiple compositions, each composition may comprise a single probiotic or a mixture of probiotics.

The compositions and methods of the present invention may further comprise one or more prebiotics.

A prebiotic is a substrate that is selectively used by a host microorganism to produce a health benefit in a subject. Without wishing to be bound by theory, prebiotics are added to nutritionally supplement bacteria in the microbiome and/or in a microbial composition, e.g., to stimulate the growth or activity of one or more strains of beneficial bacteria. Additionally, the prebiotics may be added to prevent “shock” to bacterial strains subsequent to their isolation or purification, freezing, freeze-drying, spray-drying, reconstitution in solution and the like.

Examples of prebiotics include amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

In embodiments, a prebiotic can be added (e.g., in dry or liquid forms) to a microbial composition of the present invention.

Alternately, or additionally, a prebiotic can be included (e.g., in dry or liquid forms) in a distinct pharmaceutical composition which lacks a microbial composition of the present invention.

A prebiotic may be provided to a subject before, contemporaneously with, and/or after a pharmaceutical composition comprising a microbial composition of the present invention is administered, either in a pharmaceutical composition comprising the microbial composition or in a pharmaceutical composition lacking a microbial composition.

A prebiotic may be provided in a single dose or in multiple doses. When provided as a single composition, the single composition may comprise a single prebiotic or a mixture of prebiotics. When provided in multiple compositions, each composition may comprise a single prebiotic or a mixture of prebiotics.

As examples, when multiple doses are provided, a first composition comprising a prebiotic may include one specific prebiotic, e.g., inulin, and a second composition may include a second specific prebiotic, e.g., pectin. Alternately, a first composition may include a mixture of prebiotics, e.g., inulin and pectin and a second composition may include different mixture of prebiotics, e.g., inulin and a FOS. A first composition may include a mixture of prebiotics and a second composition may include one specific prebiotic.

The amount of prebiotic provided to a subject/patient and/or included in a composition depends on the specific prebiotic, the specific bacterial strain of beneficial bacteria, and/or the disease state of the subject.

In one aspect, the present disclosure provides for determining a subject's epilepsy using one or more tests selected from the group consisting of electroencephalogram (EEG), high-density EEG, computerized tomography (CT) scan, magnetic resonance imaging (MRI), positron emission tomography (PET) scans, single-photon emission computerized tomography (SPECT), and neuropsychological tests to assess thinking, memory and speech skills. In another aspect, In one aspect, the present disclosure provides for two or more tests selected from the group consisting of electroencephalogram (EEG), high-density EEG, computerized tomography (CT) scan, magnetic resonance imaging (MRI), positron emission tomography (PET) scans, single-photon emission computerized tomography (SPECT), and neuropsychological tests to assess thinking, memory and speech skills. In an aspect, the aforementioned tests are administered prior to administering a therapeutic composition comprising a non-selected fecal microbiota. In an aspect, the aforementioned tests are administered after administering a therapeutic composition comprising a non-selected fecal microbiota.

In one aspect of the present disclosure, the subject in need thereof is non-responsive to a ketogenic diet (KD) therapy. In another aspect, the subject in need thereof is on a ketogenic diet (KD) therapy while be administered a therapeutic composition comprising a non-selected fecal microbiota.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cfu. In another aspect, a pharmaceutically active therapeutic effective dose comprises at most about 10⁵, 10⁶, 10⁷, 10⁸, 10⁹, 10¹⁰, 10¹¹, 10¹², 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cells or spores. In another aspect, a pharmaceutically active or therapeutic effective dose comprises at most about 10⁵, 10⁶, 10⁷, 10⁸, 10⁹, 10¹⁰, 10¹¹, 10¹², 10¹³, 10¹⁴, or 10¹⁵ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter. In an aspect, a pharmaceutically active or therapeutic effective dose comprises between 10¹⁰ and 10¹² cells. In another aspect, a pharmaceutically active or therapeutic effective dose comprises between 10¹⁰ and 10¹² cells per capsule. In another aspect, a pharmaceutically active or therapeutic effective dose comprises between 10¹¹ and 10¹² cells per capsule. In a further aspect, a pharmaceutically active or therapeutic effective dose comprises between 10⁹ and 10¹² cells per capsule.

In one aspect, a therapeutic composition administered herein comprises fecal bacteria. In one aspect, a therapeutic composition administered herein comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a therapeutic composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger fonnicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In one aspect, a therapeutic composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a therapeutic composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, or a combination thereof.

In one aspect, a therapeutic composition administered herein comprises a fecal microbiota. In another aspect, the preparation of a fecal microbiota used herein involves a treatment selected from the group consisting of ethanol treatment, detergent treatment, heat treatment, irradiation, and sonication. In another aspect, the preparation of a fecal microbiota used herein involves no treatment selected from the group consisting of ethanol treatment, detergent treatment, heat treatment, irradiation, and sonication. In one aspect, the preparation of a fecal microbiota used herein involves a separation step selected from the group consisting of density gradients, filtration (e.g., sieves, nylon mesh), and chromatography. In another aspect, the preparation of a fecal microbiota used herein involves no separation step selected from the group consisting of density gradients, filtration (e.g., sieves, nylon mesh), and chromatography. In another aspect, a fecal microbiota used herein comprises a donor's entire fecal microbiota. In another aspect, a therapeutic composition administered herein comprises a fecal microbiota substantially free of eukaryotic cells from the fecal microbiota's donor.

In another aspect, a therapeutic composition administered herein comprises a fecal microbiota further supplemented, spiked, or enhanced with a fecal microorganism. In one aspect, a fecal microbiota is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a therapeutic composition administered herein comprises a fecal microbiota further supplemented, spiked, or enhanced with a species of Veillonellaceae, Firmicutes, Gammaproteobacteria, Bacteroidetes, or a combination thereof. In another aspect, a therapeutic composition administered herein comprises a fecal microbiota further supplemented with fecal bacterial spores. In one aspect, fecal bacterial spores are Clostridium spores, Bacillus spores, or both.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless. In another aspect, a fecal bacteria is an uncultured population of fecal bacteria. In another aspect, a fecal bacteria is an uncultured fecal bacteria. In yet another aspect, a fecal bacteria is an uncultured collection of fecal bacteria.

In an aspect, a therapeutic composition provided or administered herein comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In another aspect, a therapeutic composition comprises fecal bacteria from at least 11, 12, 13, 14, 15, 16, 17, 18, 19, or 20 different families. In yet another aspect, a therapeutic composition comprises fecal bacteria from at least 21, 22, 23, 24, 25, 26, 27, 28, 29, or 30 different families. In a further aspect, a therapeutic composition comprises fecal bacteria from at least 31, 32, 33, 34, 35, 36, 37, 38, 39, or 40 different families. In another aspect, a therapeutic composition comprises fecal bacteria from at least 41, 42, 43, 44, 45, 46, 47, 48, 49, or 50 different families. In another aspect, a therapeutic composition comprises fecal bacteria from between 1 and 10, between 10 and 20, between 20 and 30, between 30 and 40, between 40 and 50 different families. In an aspect, a therapeutic composition provided or administered herein comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In an aspect, a pharmaceutical composition provided or administered herein comprises uncultured fecal bacteria comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a pharmaceutical composition provided or administered herein comprises uncultured fecal bacteria comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a therapeutic composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human. In an aspect, a therapeutic composition provided or administered herein comprises an extract of human feces where the composition is substantially odorless. In an aspect, a therapeutic composition provided or administered herein comprises fecal material or a fecal floral preparation in a lyophilized, crude, semi-purified or purified formulation.

In an aspect, uncultured fecal microbiota in a therapeutic composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, uncultured fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, uncultured fecal microbiota in a therapeutic composition comprises a donor's substantially entire or non-selected fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, uncultured fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, an exemplary therapeutic composition comprises starting material from a donor from a defined donor pool, where this donor contributes a stool that is centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material is separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation is now taken and filtered progressively in a filtering, e.g., a Millipore filtering or equivalent systems, to end up with liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a therapeutic composition used here comprises a reconstituted fecal flora consisting essentially of a combination of a purified fecal microbiota and a non-cellular fecal filtrate. In another aspect, a therapeutic composition used here comprises a purified fecal microbiota supplemented with one or more non-cellular non-particulate fecal components. In one aspect, a therapeutic composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based therapeutic composition is used concurrently with a fecal non-cellular filtrate-based therapeutic composition. In another aspect, a patient is treated with a first fecal non-cellular filtrate-based therapeutic composition before being given a second fecal bacteria-based therapeutic composition, or vice versa. In a further aspect, a treatment method comprises three steps: first, antibiotic pretreatment to non-selectively remove infectious pathogen(s); second, a fecal non-cellular filtrate-based treatment step to further suppress selected infectious pathogen(s); and third, giving the patient a fecal bacteria-based therapeutic composition to re-establish a functional intestinal microbiome.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of epilepsy. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377 Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastifonne, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium pilifonne, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a therapeutic composition comprises at least about 10⁵, 10⁶, 10⁷, 10⁸, 10⁹, 10¹⁰, 10¹¹, 10¹², or 10¹³ cfu or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cfu or total cell count.

In another aspect, a therapeutic composition comprises at least about 10⁵, 10⁶, 10⁷, 10⁸, 10⁹, 10¹⁰, 10¹¹, 10¹², or 10¹³ cells or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cells or total cell count.

In one aspect, a therapeutic composition is formulated as an oral capsule, microcapsule, tablet, or pill. In another aspect, a capsule, microcapsule, tablet, or pill is adapted for enteric delivery. In a further aspect, a capsule, microcapsule, tablet, or pill is an enteric capsule, microcapsule, tablet, or pill. In another aspect, a capsule, microcapsule, tablet, or pill comprises an enteric coating, is acid resistant, or both.

In one aspect, an exemplary therapeutic composition comprises starting material from a donor. In another aspect, an exemplary therapeutic composition comprises material from one or more healthy donors. In yet another aspect, an exemplary therapeutic composition comprises starting material from a defined donor pool. In another aspect, a donor is an epileptic patient in remission. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1 year old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years). In yet another aspect, a donor is a carefully screened, healthy, neurotypical human.

In an aspect, a carefully screened donor undergoes a complete medical history and physical exam. Donors are excluded if they have a risk of infectious agents. Additional exclusion criteria comprises the following:
1. Known viral infection with Hepatitis B, C or HIV
2. Known exposure to HIV or viral hepatitis at any time
3. High risk behaviors including sex for drugs or money, men who have sex with men, more than one sexual partner in the preceding 12 months, any past use of intravenous drugs or intranasal cocaine, history of incarceration.
4. Tattoo or body piercing within 12 months.
5. Travel to areas of the world where risk of traveler's diarrhea is higher than the US.
6. Current communicable disease, e.g., upper respiratory viral infection.
7. History of irritable bowel syndrome. Specific symptoms may include frequent abdominal cramps, excessive gas, bloating, abdominal distension, fecal urgency, diarrhea, constipation.
8. History of inflammatory bowel disease such as Crohn's disease, ulcerative colitis, microscopic colitis.
9. Chronic diarrhea.
10. Chronic constipation or use of laxatives.
11. History of gastrointestinal malignancy or known colon polyposis.
12. History of any abdominal surgery, e.g., gastric bypass, intestinal resection, appendectomy, cholecystectomy, etc.
13. Use of Probiotics or any other over the counter aids used by the potential donor for purpose of regulating digestion. Yogurt and kefir products are allowed if taken merely as food rather than nutritional supplements.
14. Antibiotics for any indication within the preceding 6 months.
15. Any prescribed immunosuppressive or anti-neoplastic medications.
16. Metabolic Syndrome, established or emerging. Criteria used for definition here are stricter than any established criteria. These include history of increased blood pressure, history of diabetes or glucose intolerance.
17. Known systemic autoimmunity, e.g., connective tissue disease, multiple sclerosis.
18. Known atopic diseases including asthma or eczema.
19. Chronic pain syndromes including fibromyalgia, chronic fatigue syndrome.
20. Ongoing (even if intermittent) use of any prescribed medications, including inhalers or topical creams and ointments.
21. Neurologic, neurodevelopmental, and neurodegenerative disorders including autism, Parkinson's disease.
22. General. Body mass index>26 kg/m2, central obesity defined by waste:hip ratio >0.85 (male) and >0.80 (female).
23. Blood pressure >135 mmHg systolic and >85 mmHg diastolic.
24. Skin—presence of a rash, tattoos or body piercing placed within a year, or jaundice
25. Enlarged lymph nodes.
26. Wheezing on auscultation.
27. Hepatomegaly or stigmata of liver disease.
28. Swollen or tender joints. Muscle weakness.
29. Abnormal neurologic examination.
30. Positive stool Clostridium difficile toxin B tested by PCR.
31. Positive stool cultures for any of the routine pathogens including Salmonella, Shigella, Yersinia, Campylobacter, E. coli 0157:H7.
32. Abnormal ova and parasites examination.
33. Positive Giardia, Cryptosporidium, or Helicobacter pylori antigens.
34. Positive screening for any viral illnesses, including HIV 1 and 2, Viral Hepatitis A IgM, Hepatitis surface antigen and core Ab.
35. Abnormal RPR (screen for syphilis).
36. Any abnormal liver function tests including alkaline phosphatase, aspartate aminotransaminase, alanine aminotransferase.
37. Raised serum triglycerides >150 mg/D1
38. HDL cholesterol <40 mg/dL (males) and <50 mg/dL (females)
39. High sensitivity CRP >2.4 mg/L
40. Raised fasting plasma glucose (>100 mg/dL)

In one aspect, a subject in need thereof is administered a therapeutic composition comprising fecal microbiota of multiple carefully screened, healthy donors. In an aspect, a subject is administered a therapeutic composition over a dosing period wherein a first dose comprises at least one therapeutic composition comprises fecal microbiota of a single donor, and a second dose of a therapeutic composition comprises fecal microbiota of a single donor different from the donor of the first dose. In another aspect, a first dose comprises a therapeutic composition comprising fecal microbiota of a single donor and a second dose comprises fecal microbiota of a donor pool. The first and second dose do not indicate the order of administration to a subject, but rather that fecal microbiota from separate donors may be used in a non-blended form.

In another aspect, the present disclosure provides for methods for treating a subject in need thereof with capsules containing a therapeutic composition comprising fecal microbiota from a single donor. In another aspect, a capsule comprises a therapeutic composition comprising fecal microbiota from multiple donors. In one aspect a subject is administered two or more pills comprising fecal microbiota from a single but different donor.

In one aspect, the present disclosure provides for methods for treating a subject in need thereof comprising administering a therapeutic composition orally or by infusions through a colonoscope, an enema or via a nasojejunal tube. In another aspect, each administration comprises a therapeutic composition comprising fecal microbiota of a single donor similar to or different from a prior administration in a treatment period. In another aspect, a treatment period includes administration of a first dost comprising a therapeutic composition comprising fecal microbiota of a single donor and administration of a second dose comprising a therapeutic composition comprising fecal microbiota of multiple donors.

In one aspect, the present disclosure provides for the following embodiments:

Embodiment 1. A method for treating epilepsy or seizure in a subject in need thereof, said method comprising administering to said subject an oral capsule comprising a pharmaceutically active dose of a therapeutic composition comprising a non-selected fecal microbiota.

Embodiment 2. The method of embodiment 1, wherein said seizure is selected from the group consisting of focal or generalized seizures.

Embodiment 3. The method of embodiment 2, wherein said focal seizure is selected from the group consisting of simple focal seizure, complex focal seizure, and secondary generalized seizure.

Embodiment 4. The method of embodiment 2, wherein said generalized seizure is selected from the group consisting of absence, tonic, atonic, clonic, myoclonic, or tonic-clonic seizures.

Embodiment 5. The method of embodiment 1, wherein said composition comprises an isolated or purified population of said live non-pathogenic fecal bacteria.

Embodiment 6. The method of embodiment 1, wherein said method reduces the overall frequency of seizures.

Embodiment 7. The method of embodiment 6, wherein said frequency is reduced per day, per week, per month, or per year.

Embodiment 8. The method of embodiment 1, wherein said methods prevent a relapse in seizures.

Embodiment 9. The method of embodiment 1, wherein said method reduces the frequency of warnings that an epileptic seizure is about to occur.

Embodiment 10. The method of embodiment 7, wherein said method reduces said frequency by at least 10%, 20%, 30%, 50%, 60%, 70%, 80%, or 90% after 4, 8, or 12 weeks of treatment.

Embodiment 11. The method of embodiment 1, wherein said administration is on a daily or weekly basis.

Embodiment 12. The method of embodiment 1, wherein said administration lasts at least 1, 2, 3, 4, 5, 6, 7, 8, 9, or 10 weeks.

Embodiment 13. The method of embodiment 1, wherein said dose is administered at least once daily or weekly for at least 2, 3, 4, 5, 6, 7, 8, 9, 10, 11, 12, 13, 14, or 15 consecutive days.

Embodiment 14. The method of embodiment 1, wherein said dose is administered at least once daily or weekly for at least 1, 2, 3, 4, 5, 6, 7, 8, 9, 10, 11, or 12 consecutive weeks.

Embodiment 15. The method of embodiment 1, wherein said dose is administered at least once daily or weekly for at most 3, 4, 5, 6, 7, 8, 9, 10, 11, 12, 13, 14, 15, 16, 17, 18, 19, or 20 consecutive days.

Embodiment 16. The method of embodiment 1, wherein said dose is administered at least once daily or weekly for at most 1, 2, 3, 4, 5, 6, 7, 8, 9, 10, 11, or 12 consecutive weeks.

Embodiment 17. The method of embodiment 1, wherein said dose is administered at least twice daily or weekly for at least two consecutive days.

Embodiment 18. The method of embodiment 17, wherein said dose is administered at least twice daily or weekly for at least 3, 4, 5, 6, 7, 8, 9, 10, 11, 12, 13, 14, or 15 consecutive days.

Embodiment 19. The method of embodiment 17, wherein said dose is administered at least twice daily or weekly for at least 1, 2, 3, 4, 5, 6, 7, 8, 9, 10, 11, or 12 consecutive weeks.

Embodiment 20. The method of embodiment 17, wherein said dose is administered at least twice daily or weekly for at most 3, 4, 5, 6, 7, 8, 9, 10, 11, 12, 13, 14, 15, 16, 17, 18, 19, or 20 consecutive days.

Embodiment 21. The method of embodiment 17, wherein said dose is administered at least twice daily or weekly for at most 1, 2, 3, 4, 5, 6, 7, 8, 9, 10, 11, or 12 consecutive weeks.

Embodiment 22. The method of embodiment 17, wherein said dose is administered at least three times daily for at least one day.

Embodiment 23. The method of embodiment 1, wherein said capsule is an enteric coated capsule, an acid-resistant, enteric-coated capsule, or an enteric coated microcapsule.

Embodiment 24. The method of any one of the preceding embodiments, wherein said method further comprises administering one or more medications selected from the group consisting of carbamazepine, clobazam, diazepam, divalproex, eslicarbazepine acetate, ethosuximide, gabapentin, lacosamide, methsuximide, oxcarbazepine, perampanel, phenobarbital, phenytoin, pregabalin, refinamide, tiagabine hydrochloride, vigabatril, and a combination thereof.

Embodiment 25. The method of any one of the preceding embodiments, wherein said method further comprises administering one or more medications selected from the group consisting of clonazepam, clorazepate, ezogabine, felbamate, lamotrigine, levetiracetam, lorazepam, primidone, topiramate, valproic acid, zonisamide and a combination thereof.

Embodiment 26. A method of treating epilepsy or seizure in a subject in need thereof, said method comprising orally administering to said subject a pharmaceutically active dose of a therapeutic composition comprising a non-selected fecal microbiota.

Embodiment 27. The method of embodiment 26, wherein said composition is in a liquid, frozen, freeze-dried, spray-dried, foam-dried, lyophilized, or powder form.

Embodiment 28. The method of embodiment 26, wherein said composition is in a capsule.

Embodiment 29. The method of embodiment 26, wherein said subject is pretreated with an antibiotic prior to administration of said composition.

Embodiment 30. The method of embodiment 29, wherein said antibiotic is selected from the group consisting of rifabutin, clarithromycin, clofazimine, vancomycin, rifampicin, nitroimidazole, chloramphenicol, and a combination thereof.

Embodiment 31. The method of embodiment 29, wherein said antibiotic is selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof.

Embodiment 32. The method of embodiment 26, wherein said administration comprises an induction treatment and a maintenance treatment.

Embodiment 33. The method of embodiment 32, wherein said induction period comprises administering a greater number of capsules per day than said maintenance treatment.

Embodiment 34. The method of embodiment 26, wherein said subject in need thereof is deficient in one or more of Akkermansia or Parabacteroides species relative to a subject who does not have epilepsy.

Embodiment 35. The method of embodiment 26, wherein said subject in need thereof is non-responsive to ketogenic diet (KD) therapy.

Embodiment 36. The method of embodiment 26, wherein said subject in need thereof is pretreated with a probiotic prior to administration of said composition.

Embodiment 37. The method of embodiment 26, wherein said administering further comprises co-administering a probiotic.

Embodiment 38. The method of embodiment 36 or 37, wherein said probiotic is selected from the group consisting of Akkermansia or Parabacteroides species.

Embodiment 39. The method of embodiment 26, wherein said subject in need thereof is also on ketogenic diet (KD) therapy.

Embodiment 40. The method of embodiment 26, further comprising examining said subject in need thereof prior to said administering

Embodiment 41. The method of embodiment 40, wherein said examining is a test selected from the group consisting of electroencephalogram (EEG), high-density EEG, computerized tomography (CT) scan, magnetic resonance imaging (MRI), positron emission neuropsychological tests to assess thinking, memory and speech skills.

Embodiment 42. The method of embodiment 1, wherein said administration is on a daily or weekly basis.

Embodiment 43. The method of embodiment 26, wherein said administration lasts at least 1, 2, 3, 4, 5, 6, 7, 8, 9, or 10 weeks.

Embodiment 44. The method of embodiment 26, wherein said dose is administered at least once daily or weekly for at least 2, 3, 4, 5, 6, 7, 8, 9, 10, 11, 12, 13, 14, or 15 consecutive days.

Embodiment 45. The method of embodiment 26, wherein said dose is administered at least once daily or weekly for at least 1, 2, 3, 4, 5, 6, 7, 8, 9, 10, 11, or 12 consecutive weeks.

Embodiment 46. The method of embodiment 26, wherein said dose is administered at least once daily or weekly for at most 3, 4, 5, 6, 7, 8, 9, 10, 11, 12, 13, 14, 15, 16, 17, 18, 19, or 20 consecutive days.

Embodiment 47. A method of treating epilepsy or seizure in a subject in need thereof, said method comprising determining the relative abundance of one or more Akkermansia or Parabacteroides species in an intestine of the subject and orally administering to said subject a pharmaceutically active dose of a therapeutic composition comprising a non-selected fecal microbiota if the relative abundance of the one or more Akkermansia or Parabacteroides species is less than a threshold level.

Embodiment 48. The method of embodiment 47, wherein said testing further comprises examining said patient with electroencephalogram (EEG), high-density EEG, computerized tomography (CT) scan, magnetic resonance imaging (MRI), positron emission tomography (PET) scans, single-photon emission computerized tomography (SPECT), and neuropsychological tests to assess thinking, memory and speech skills.

Embodiment 49. A method of treating epilepsy or seizure in a subject in need thereof, said method comprising determining the relative abundance of one or more Clostridiales, Ruminococcaceae, Rikenellaceae, Lachnospiraceae, and Alistipes species in an intestine of the subject and orally administering to said subject a pharmaceutically active dose of a therapeutic composition comprising a non-selected fecal microbiota.

Embodiment 50. The method of embodiment 49, wherein said testing further comprises examining said patient with electroencephalogram (EEG), high-density EEG, computerized tomography (CT) scan, magnetic resonance imaging (MRI), positron emission neuropsychological tests to assess thinking, memory and speech skills.

Embodiment 51. A method of treating epilepsy or seizure in a subject in need thereof, said method comprising determining the relative abundance of one or more Proteobacteria, Cronobacter, Bacteroides, Prevotella, and Bifidobacterium species in an intestine of the subject and orally administering to said subject a pharmaceutically active dose of a therapeutic composition comprising a non-selected fecal microbiota.

Embodiment 52. The method of embodiment 51, wherein said testing further comprises examining said patient with electroencephalogram (EEG), high-density EEG, computerized tomography (CT) scan, magnetic resonance imaging (MRI), positron emission tomography (PET) scans, single-photon emission computerized tomography (SPECT), and neuropsychological tests to assess thinking, memory and speech skills.

Embodiment 53. A method of treating epilepsy or seizure in a subject in need thereof, said method comprising orally administering to said subject a pharmaceutically active dose of a therapeutic composition comprising a non-selected fecal microbiota, wherein said administering comprises at least 10 capsules.

Embodiment 54. A method of treating epilepsy or seizure in a subject in need thereof, said method comprising administering to said subject a pharmaceutically active dose of a therapeutic composition comprising a non-selected fecal microbiota, wherein said subject is free of gut dysbiosis immediately prior to said treating.

Embodiment 55. A method of treating epilepsy or seizure in a subject in need thereof, said method comprising administering to said subject a pharmaceutically active dose of a therapeutic composition comprising a non-selected fecal microbiota, wherein said subject does not exhibit signs of malnutrition or growth retardation.

Embodiment 56. A method of treating epilepsy or seizure in a subject in need thereof, said method comprising administering to said subject an antibiotic and a pharmaceutically active dose of a therapeutic composition comprising a non-selected fecal microbiota.

Embodiment 57. The method of embodiment 56, wherein said antibiotic is Vancoymcin or Rifaximin.

Embodiment 58. A method of treating epilepsy or seizure in a subject in need thereof, said method comprising administering to said subject a pharmaceutically active dose of a first and second therapeutic composition, wherein said first and second therapeutic composition each comprise a non-selected fecal microbiota.

Embodiment 59. The method of embodiment 58, wherein at least one of said first and second therapeutic composition comprises an oral capsule.

Embodiment 60. The method of embodiment 58, wherein said first therapeutic composition comprises a non-selected fecal microbiota from a first donor and said second therapeutic composition comprises a non-selected fecal microbiota from a second donor.

Embodiment 61. The method of embodiment 60, wherein said first and second therapeutic compositions are administered to said subject on different days.

Embodiment 62. The method of embodiment 58, wherein at least one of said first and second therapeutic composition comprises a non-selected fecal microbiota from a donor pool.

The disclosure may be better understood by reference to the following non-limiting Examples, which are provided as exemplary of the disclosure. The following examples are presented in order to more fully illustrate the preferred aspects of the disclosure and should in no way be construed, however, as limiting the broad scope of the disclosure. Therefore, the scope of the appended claims should not be limited to the description of the aspects contained herein.

### Examples

### Example 1. Preparation of Fecal Microbiota

Fecal microbiota is prepared essentially according to protocols published in US2014/0147417 or WO2014/152484. Summarized below is an exemplary protocol.

Potential fecal microbiota donors are screened according to a list of criteria used to exclude unsuitable donors. Potential fecal microbiota donors are excluded if they have received antibiotics, laxatives, diet pills, immunomodulators or chemotherapy in the preceding three months. Potential fecal microbiota donors are excluded if they have a history of all known infectious diseases, morbid obesity, diabetes, irritable bowel syndrome, inflammatory bowel disease, chronic diarrhea, constipation, colorectal polyps or cancer, a compromised immune system, metabolic syndromes, chronic fatigue syndrome, major GI surgery, or other diseases or conditions potentially associated with specific changes in fecal microbiota. Potential fecal microbiota donors are excluded if they exhibit positive laboratory tests for C-reactive protein, erythrocyte sedimentation rate, hepatitis A, hepatitis B, hepatitis C, human immunodeficiency virus, human T-lymphotropic virus, or syphilis. Potential fecal microbiota donors are excluded if they exhibit a positive test for stool ova, parasites, and/or viruses. Potential fecal microbiota donors are excluded if they engage in high-risk sexual behaviors, have been incarcerated, or received any tattoos or body piercings in areas that have had disease epidemics within the past three months.

Donor fecal material (fresh feces) is collected in a sterilized container, and then it is transferred to a blender. Approximately 500-1000 mL 0.9% saline solution is added to the blender and thoroughly mixed with the fecal sample. The resulting suspension is filtered at least 4 times through strainers prior to collecting a final suspension. The final suspension is centrifuged in 50 mL tubes at 1200×g for 3 minutes. The supernatant is discarded and the pellet is gently resuspended in approximately 50 mL of sterile 0.9% saline solution. The centrifugation and resuspension steps are repeated 2 to 4 additional times. Upon the final centrifugation, the supernatant is discarded. If the fecal microbiota is to be used immediately, the resultant pellet is resuspended in 1.5-volumes of 0.9% saline solution by gently mixing. If the fecal microbiota is to be stored, the resultant pellet is resuspended in 10% sterile glycerol and stored at −80 degrees Centigrade. If fecal microbiota are frozen, they are warmed to room temperature prior to administration to a patient

### Example 2. Treatment of Epilepsy

A 31 year old male patient with a long history of seizures and convulsions is treated with fecal microbiome therapy. The patient is initially diagnosed with complex partial seizures with secondary generalization, which progress to grand mal seizures. Anti-convulsive therapy is prescribed since childhood and continues during fecal microbiome therapy. The patient does not experience any growth retardation or malnutrition as a result of the seizures. The patient also suffered from ileitis and utilized a treatment plan of Vancomycin and Rifaximin, which reduces convulsions. Prior to the fecal microbiome therapy, the patient suffers approximately 5 seizures per day. Multiple daily seizures have been present since childhood. The recovery time from seizures can be over one hour. The patient experiences an aura stage which preempts a seizure. Fecal microbiome therapy is administered as 10 infusions. After treatment, overall frequency of seizures is decreased and recovery time from seizures is reduced to 30 minutes or less. One month after experiencing a seizure, the patient is administered capsule fecal microbiome therapy at one capsule per day. The patient experiences a seizure on day 1, day 34, and day 70 after beginning capsule therapy. Otherwise, patient experiences aura stage but no seizures. While on capsule therapy, aura duration and frequency significantly decreases. On day 78 after beginning treatment the patient experiences 9 aura episodes. The patient begins a new batch of capsules from a different donor. The aura episodes decreases to 6 episodes per day, 3 episodes per day, and 2 episodes per day, 1, 2, and 3, days after beginning the new batch, respectively. Aura episodes eventually cease. The patient's epilepsy symptoms progressively improve while on capsule treatment.

### Example 3. Treatment of Epilepsy

A 32-year old male patient with an 18 year history of grand mal seizures is treated with fecal microbiome therapy. The patient's fits are of varying severity depending to a certain extent on his diet. A neurologist is unable to determine a cause for the patient's fits. The patient initially presents with diarrhea, cramping, and the presence of clostridia, and is treated with Vancomycin 250 ii b.i.d. Vancomycin treatment reverses the epilepsy when the subject is taking the Vancomycin. This suggests that the epilepsy is at least partially driven by a portion of the patient's gut microbiota which is reduced during Vancomycin treatment. The patient is then treated with orally administered capsules containing lyophilized fecal material—a type of oral fecal microbiota transplantation (FMT). A single capsule contains between 10⁹ and 10¹² viable cells and the patient ingests initially 8 capsules per day, 4 with breakfast and 4 with dinner, later reducing to 2 capsules twice daily. After administration of adequate doses of the capsules the patient's epilepsy is completely suppressed for many months. Upon dosage reduction to 2 capsules per day, minor forms of fits begin to reoccur. The patient is then treated with liquid fecal microbiome transplantation using 300 ml of homogenized, filtered fresh donor stool. The patient is now free of epileptic fits for 7 months.

### Example 4. Oral Capsule Treatment Protocol for Epilepsy

Patients are divided into four groups (Groups 1 to 4). Group 1 patients are administered a pretreatment of antibiotics (e.g., Vancomycin and Metronidazole). Group 2 receives no antibiotics. Both Groups 1 and 2 receive a pre-colonoscopy bowel prep followed by capsule fecal microbiome therapy. Groups 3 and 4 receive no bowel prep while Group 3, but not Group 4, also receives an antibiotic pretreatment. A single capsule contains between 10⁹ and 10¹² viable cells. Capsules are administered for 18 weeks as follows: two capsules twice-a-day for 14 days, two capsules twice-a-day every other day for 14 days, 4 capsules twice-a-week for 14 days, and 4 capsules once-a-week (e.g., each Monday) for 12 weeks. High dose capsules (total cell count of about 10¹²) are used in loading doses (also called treatment doses) for the initial 4 weeks. Lower dose capsules (total cell count of about 10⁹) are used in maintenance doses for the subsequent 14 weeks. In patients receiving antibiotic pretreatment, capsules are administered one day after ceasing antibiotics. Patient symptoms are observed and clinical examination is performed before, during and post oral capsule treatment. Pre, during and post-treatment DNA metagenomics (2-4 days; 1 week; 6 weeks; 12 weeks) of patient fecal samples are also carried out. The capsule treatments reverse patient symptoms of seizures. Patients receiving capsule treatment also experience a clinically normal urge and defecation.

As various modifications could be made in the constructions and methods herein described and illustrated without departing from the scope of the disclosure, it is intended that all matter contained in the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entireties.

1. A method for treating epilepsy or seizure in a subject in need thereof, said method comprising orally administering to said subject a pharmaceutically active dose of a therapeutic composition comprising uncultured fecal bacteria.

2. The method of claim 1, wherein said seizure is selected from the group consisting of focal or generalized seizures.

3. The method of claim 2, wherein said focal seizure is selected from the group consisting of simple focal seizure, complex focal seizure, and secondary generalized seizure.

4. The method of claim 2, wherein said generalized seizure is selected from the group consisting of absence, tonic, atonic, clonic, myoclonic, or tonic-clonic seizures.

5. The method of claim 1, wherein said method reduces the overall frequency of seizures.

6. The method of claim 1, wherein said methods prevent a relapse in seizures.

7. The method of claim 5, wherein said method reduces said frequency by at least 10%, 20%, 30%, 50%, 60%, 70%, 80%, or 90% after 4, 8, or 12 weeks of treatment.

8. The method of claim 1, wherein said administration is on a daily or weekly basis.

9. The method of claim 1, wherein said composition is in a capsule.

10. The method of claim 1, wherein said composition is in a liquid, frozen, freeze-dried, spray-dried, foam-dried, lyophilized, or powder form.

11. The method of claim 1, wherein said capsule is an enteric coated capsule, an acid-resistant, enteric-coated capsule, or an enteric coated microcapsule.

12. The method of claim 1, wherein said subject is pretreated with an antibiotic prior to administration of said composition.

13. The method of claim 1, wherein said administration comprises an induction treatment and a maintenance treatment.

14. The method of claim 13, wherein said induction period comprises administering a greater number of capsules per day than said maintenance treatment.

15. The method of claim 1, wherein said subject in need thereof is deficient in one or more of Akkermansia or Parabacteroides species relative to a subject who does not have epilepsy.

16. The method of claim 1, wherein said subject in need thereof is non-responsive to ketogenic diet (KD) therapy.

17. A method of treating epilepsy or seizure in a subject in need thereof, said method comprising determining the relative abundance of one or more Akkermansia or Parabacteroides species in an intestine of the subject and orally administering to said subject a pharmaceutically active dose of a therapeutic composition comprising a non-selected fecal microbiota if the relative abundance of the one or more Akkermansia or Parabacteroides species is less than a threshold level.

18. A method of treating epilepsy or seizure in a subject in need thereof, said method comprising administering to said subject a pharmaceutically active dose of a therapeutic composition comprising uncultured fecal bacteria, wherein said subject is free of gut dysbiosis immediately prior to said treating.

19. The method of claim 18, wherein said composition is in a capsule.

20. The method of claim 18, wherein said composition is in a liquid, frozen, freeze-dried, spray-dried, foam-dried, lyophilized, or powder form.
